(19) United States
(12) Patent Application Publication
(10) Pub. No.: US 2005/0004602 A1
(43) Pub. Date: Jan. 6, 2005

(54) Interlocking suture clinch

(75) Inventors: Charles C. Hart, Summerville, SC (US); Matthew A. Wixey, Dana Point, CA (US)

Correspondence Address: Kenneth K. Vu, 22872 Avenida Empresa, Rancho Santa Margarita, CA, 92688, US

(73) Assignee: Applied Medical Resources Corporation

(21) Appl. No.: 10/612,631

(22) Filed: Jul. 2, 2003

(51) Int. Cl. A61B 17/04

(52) U.S. Cl. 606/232

(57) ABSTRACT

The invention is directed to a suture clinch that may be placed upon suture extensions of a placed suture to securely lock the extensions in a desired location. The clinch operates to replace a tied knot and retain the suture in a fixed relationship with little or no damage to the suture itself. The clinch comprises a mating pair of interlocking members that restrict movements of the suture. The interlocking members may be placed upon the suture extensions from alongside the suture extensions and compressed to a first condition where the interlocking members may be advanced, retracted or adjusted, and subsequently further compressed to a second condition where the interlocking members are fully engaged to fully restrict the movement of the suture. In another aspect, the clinch may be provided in a flat form comprising a first component and a second component. The first and second components fit together in a mating relationship that entraps at least a portion of the suture between tractive faces of the first and second components, respectively. Each end of the first and second components may have a recessed central portion that leaves two extensions along the sides of each end of the components. The extensions of the second component are subsequently folded over the first component to provide secure entrapment of the suture between the tractive faces of the first and second components. The tractive faces operate to engage the suture and hold it securely in place without damaging or challenging the suture. The clinch may comprise of plastic, metallic materials such as stainless steel or titanium, or a combination of both where the plastic is chosen for its value as an atraumatic interface and the metal for its radiopacity or durability.

### BACKGROUND OF THE INVENTION

1. Field of the Invention

This invention generally relates to surgical clips and clamps and, in particular, to clinches adapted for use with sutures.

2. Discussion of the Relevant Art

Clips, clamps and surgical sutures are commonly used in both open and minimally invasive surgery to hold objects such as tissues, vessels and other surgical devices in close proximity. When sutures are used, the most common way to secure the extending ends of a placed suture is to tie a knot. There are many different types of knots used to tie the extending ends. Some knots are designed to provide instant security while others are designed to slip into place. It is appreciated that a tied knot should remain in place without slipping or becoming un-tied or undone. Placing and tying a secured knot, however, has proved to be a real challenge for many surgeons because of the time and skill that are required to perform this task, especially in minimally invasive or laparoscopic surgery. In particular, the task of placing and tying a knot with elongated graspers while looking into a video monitor is a challenge even for the most gifted and talented surgeons. In order to place and keep a knot in a desired location, surgeons have had to loosely tie the extending ends of a suture in a complex combination of “throws” or square knots, then advance the combination to the desired location and subsequently tighten or secure the placed knot combination. That is, it is not uncommon to see from six to ten throws, or individual square knots, stacked upon each other to secure a single pair of suture ends. This method, of course, takes valuable time and does not make the best use of a surgeon's time or skill.

Moreover, a tied surgical knot has mechanical and physical problems that must be dealt with. For example, the suture material must be able to withstand the placement of such knot without chafing, elongating, binding, twisting or breaking. In many cases, the suture is selected to meet the challenge of placement security rather than what is most appropriate for a surgical application. Another challenge is the multi-throw knot that is placed upon a pair of suture ends to be advanced and tightened at a desired location must have a small diameter. Small diameter sutures such as 3-0 to 4-0 gage sutures, however, will likely be damaged as a knot advances along the suture ends. In addition, as the knot is tightened by surgical devices such as graspers or “knot-pushers,” the suture is subject to elongation and compression. This results in a knot having compressed radii over that portion of the suture that must extend. It is known in the art that the size of the radii over which the suture extends has a dramatic bearing on the security of any given suture placement. Specifically, a knot that is excessively tightened may be weaker than one that is more loosely tightened, but then the more loosely tightened one may be subject to slippage.

Accordingly, there is a need in the art for a suture securing device that replaces the tied knot. Several attempts have been made but have met with limited success. For example, a foldable suture clinch has been used to compress upon a pair of suture ends. While this arrangement works to securely hold the suture, it suffers from problems associated with the radius postulate. Moreover, the clinch requires a lot of space to fold the ends of the clinch around the suture. Attempts have also been made to place a cylindrical device upon the suture ends, which is very difficult to do, as the cylindrical device must be placed over the suture ends as the ends are held in traction. As such, various clips and clamps have been disclosed and tried, but all have met with only limited success.

### SUMMARY OF THE INVENTION

The present invention is directed to a suture-securing device such as a clinch that may be placed upon suture extensions of a placed suture to securely lock the extensions in a desired location. The clinch operates to replace a tied knot and retain the suture in a fixed relationship with little or no damage to the suture itself. The clinch comprises a mating pair of interlocking members that restrict movements of the suture. The interlocking members may be placed upon the suture extensions from alongside the suture extensions and compressed to a first condition where the interlocking members may be advanced, retracted or adjusted, and subsequently further compressed to a second condition where the interlocking members are fully engaged to fully restrict the movement of the suture.

In another aspect of the invention, a suture clinch may be provided in a flat form comprising a first component and a second component. The first and second components are sized and configured to fit together in a mating relationship that entraps at least a portion of material such as surgical suture between tractive faces of the first and second components, respectively. Subsequent folding of the second component over the first component provides secure entrapment of material between the tractive faces of the first and second components. More specifically, the tractive faces are sized and configured to engage surgical suture and hold it securely in place without damaging or challenging the suture. Each end of the first and second components may have a recessed central portion that leaves two extensions along the sides of each end. The extensions of the second component are subsequently folded over the first component to provide secure entrapment of the suture.

The interlocking nature of the first and second components of the invention provides entrapment of surgical suture or other elongate material in a unique combination. First, the entrapped material is held between two opposing tractive faces that may have traction-enhancing features. However, the entrapped material is not crushed to the extreme that would be required without the traction-enhancing features. It is important to provide stability and security to the entrapped material without providing excessive pressure or stress on the delicate material. As such, a raised crosscut pattern having very small, sharp details is preferred. The centrally located raised pattern allows the entrapped material to move within the recesses associated with the second component without abrasion. Any motion associated with suture extensions is isolated within the recessed region. The first and second components of the invention lightly compress the entrapped suture between the tractive faces and provide rounded material edges that are sized and configured to maintain the intrinsic strength of the suture as it exerts a “cross-pull” against the reinforced folded extensions of the second component.

The clinches of the invention may comprise of plastics that are of the same genus as the suture itself. This will take advantage of the tractive relationship between like plastic materials. Other materials include biodegradable plastics such as polyglycolic acid or polylactate materials that have a rate of absorption commensurate with the suture used. Metallic materials such as stainless steel, titanium or rare metals may also be used. In alternate embodiments, a combination of metal and plastic may also be used where the plastic is chosen for its value as an atraumatic interface and the metal for its radiopacity or durability.

These and other features and advantages of the invention will become more apparent with a discussion of preferred embodiments in reference to the associated drawings.

### DESCRIPTION OF THE DRAWINGS

FIGS. 1(A) and 1(B) illustrate a complex slip-knot and a tied multi-throw surgical knot, respectively, used in laparoscopic surgery;

FIG. 2 is a perspective view of a suture clinch with interlocking members snapped together in accordance with the first embodiment of the invention;

FIG. 3 is a perspective view of the front face of an interlocking member of the invention;

FIG. 4 is a side view of the interlocking member of the invention;

FIG. 5 is a back view of the interlocking member of the invention;

FIGS. 6(A) and 6(B) are a first end view and a second end view, respectively, of the interlocking member of the invention;

FIG. 7 illustrates the path of the suture through the interlocking members of the invention;

FIGS. 8(A)-8(C) illustrate the suture and radii relationship of the radius postulate;

FIG. 9 is a perspective view of a suture clinch and clinch applier operatively positioned for use in a patient;

FIG. 10 is a perspective view of the interlocking members of the invention in a first condition;

FIG. 11 is a perspective view of the interlocking members of the invention in a second condition;

FIG. 12 is a perspective view of the interlocking members of the invention in a final condition;

FIG. 13 illustrates another embodiment of the interlocking members of the invention made from a metallic material;

FIG. 14 is a perspective view of the front face of a metallic interlocking member of the invention;

FIG. 15 is a perspective view of two metallic interlocking members in a first condition;

FIG. 16 is a perspective view of two metallic interlocking members in a second condition;

FIG. 17 is a perspective view of two metallic interlocking members in a final condition;

FIG. 18 illustrates a suture clinch in accordance with another embodiment of the invention in a flat form;

FIG. 19 illustrates the suture clinch of FIG. 18 in a mating relationship prior to folding;

FIG. 20 illustrates the suture clinch of FIG. 18 in a mating relationship after initial folding;

FIG. 21 is a top/perspective view of the assembled and folded suture clinch of FIG. 18;

FIG. 22 is a top view of the assembled and folded suture clinch of FIG. 18;

FIG. 23 is a perspective end view of a clinch applier associated with the present invention;

FIG. 24 is a perspective side view of the clinch applier associated with the invention;

FIG. 25 is a top view of the clinch applier associated with the invention;

FIG. 26 is a front end view of the clinch applier associated with the invention; and

FIG. 27 is a cut-away view of the distal portion of the clinch applier.

### DESCRIPTION OF PREFERRED EMBODIMENT AND BEST MODE OF THE INVENTION

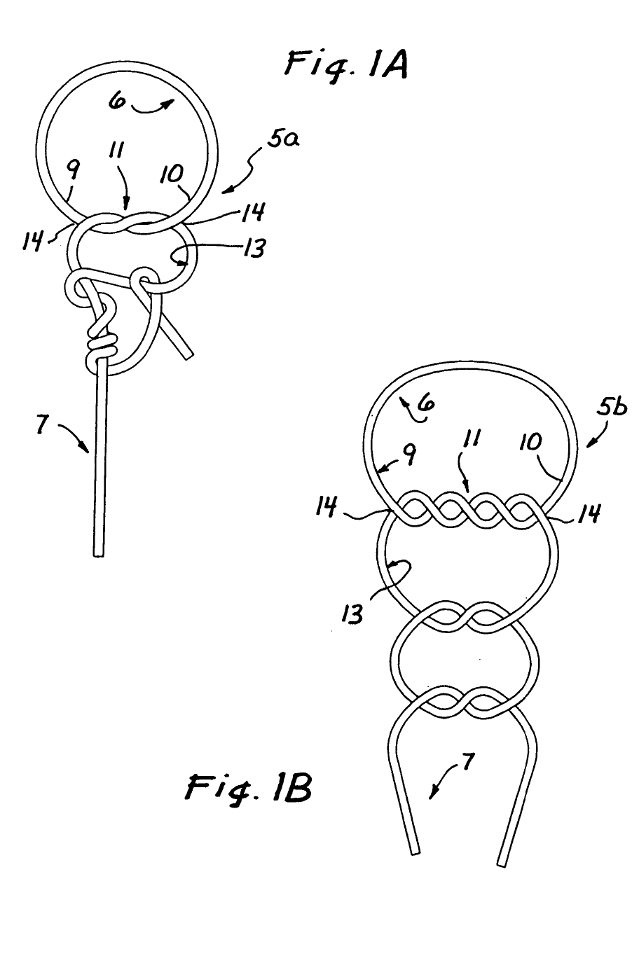

FIGS. 1(A) and 1(B) illustrate a complex slip-knot 5a and a multi-throw surgical knot 5b, respectively, used in laparoscopic surgery having an engaging portion 6 and an extending portion 7. The engaging portion 6 passes through a body tissue of a patient to be approximated and the extending portion 7 is secured to hold the engaging portion 6 in place. Most often, the engaging portion 6 comprises a generally circular configuration where portions 9, 10 of the suture that exit the distal end 11 of the tied knots 5a and 5b are nearly one hundred and eighty degrees (180°) apart. The most common configuration presents a cross-pull against either of the knots 5a and 5b. The radius of suture 13 becomes the radius over which the suture 13 must pass. This relationship will be referred to as the radius postulate. Plainly stated the radius postulate says that the radius over which any given suture passes shall be no less than the radius of the suture itself. Extrapolating this principle provides that a larger radius adds an element of safety to the securement of a suture. As a tied knot is tightened, compression of the material is inevitable and the radii of the suture at critical cross-points 14 are reduced. In addition, stress, friction and elongation combine to make the tied knots 5a and 5b the weakest point in the surgical suture loop. Breakage of suture material typically occurs either as the knot is being tied or at the distal exit 11 of the tied knot. Suture choice is often mitigated by this condition.

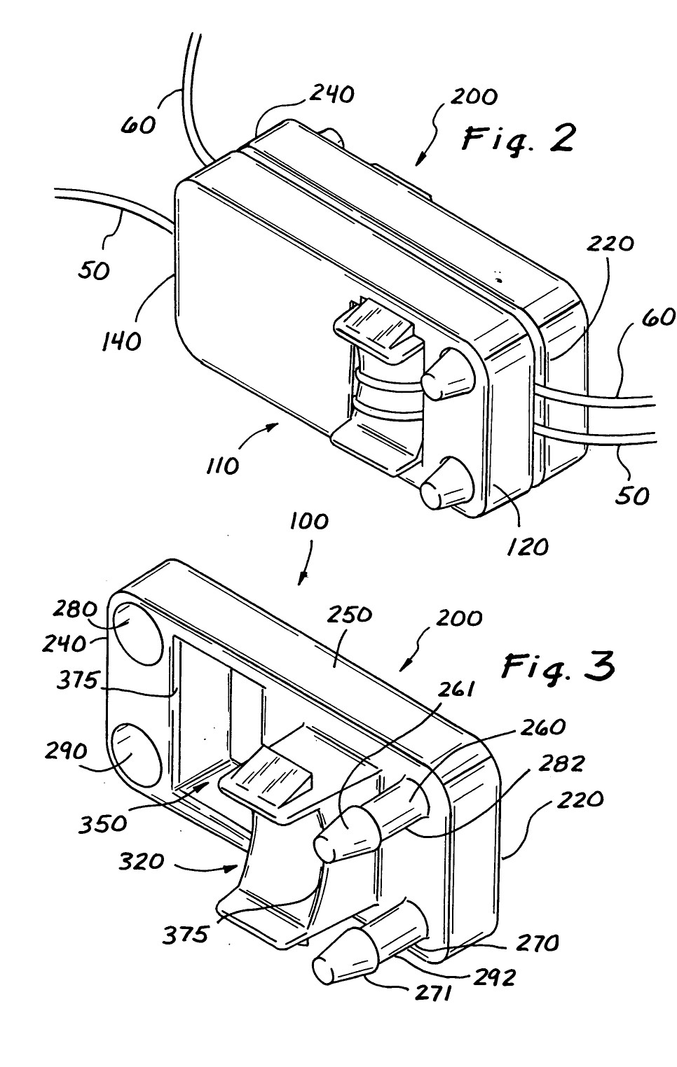

Referring to FIG. 2, there is a shown a perspective view of a suture clinch 100 in accordance with a first embodiment of the invention. Suture clinch 100 comprises a first interlocking member 110 and a second interlocking member 200 that operate to snap together to securely lock suture extensions 50, 60 in a desired location. More particularly, FIG. 2 illustrates the interlocking members 110, 200 as the interlocking members are fully compressed and locked together in a final condition. Two extensions of suture 50, 60 are seen to pass through the interlocking members 110, 200 from proximal ends 120, 220 to distal ends 140, 240 of the interlocking members 110, 200, respectively. The suture 50, 60 is trapped in a convoluted path and forced into portions of the interlocking members that prevent the suture 50, 60 from moving in any direction.

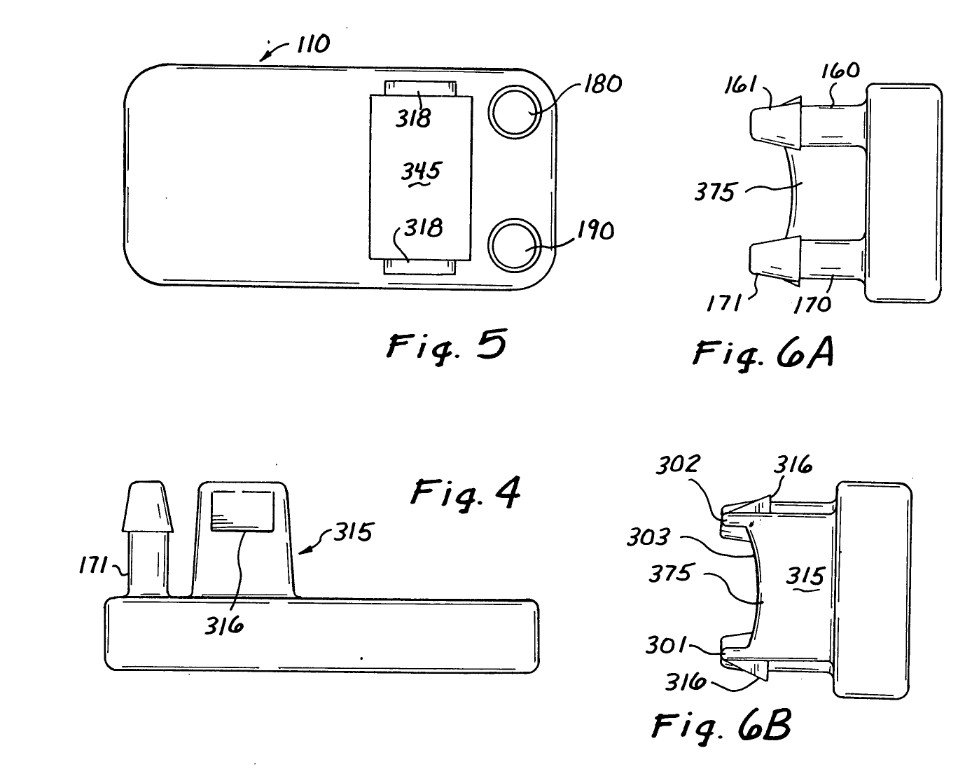

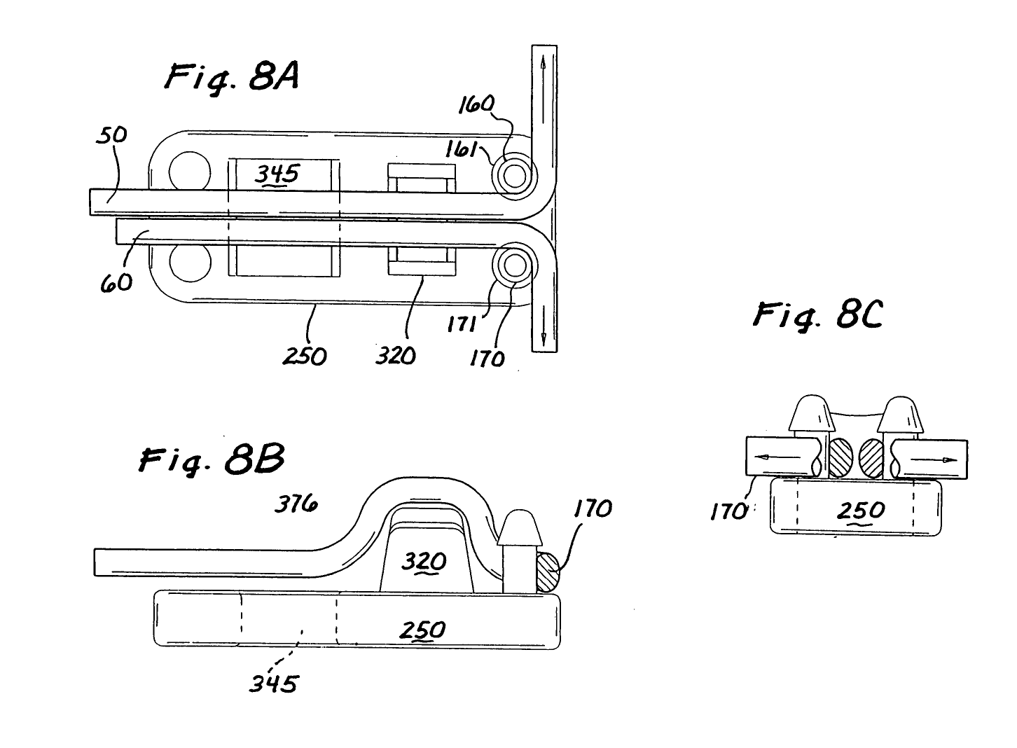

FIGS. 3-7 illustrate one of the interlocking members where there is a generally rectangular base 250 having a length, a width and a thickness. Upon the base 250, at one end, there are cylindrical protrusions 260, 270 sized and configured to match countersunk holes 280, 290 at the opposite end of the base 250. The protrusions 260, 270 may have, in a preferred embodiment, barbs 261, 271 or increased end diameter to engage the countersunk holes 280, 290 in a fixed relationship when fully mated. Of particular interest is the radius 282, 292 of the protrusions 260, 270 where the radius 282, 292 is as large as possible for the size of the base 250 and the mating holes 280, 290. The protrusions 260, 270 are the radii over which the suture 50, 60 must pass since the suture 50, 60 will always align with the base 250 and oppose the protrusions 260, 270. Upon one half of the base 250 there is a standing portion 320 sized and configured to engage and confine the suture extensions 50, 60. Within the other half of the base is a window 350 sized and configured to receive the standing portion 320 and engage and confine suture 50, 60.

When the two interlocking members 110, 200 are alternately approximated, the included suture 50, 60 is trapped between four protrusions 160, 170, 260, 270 and forced over two standing portions 315, 320 and into two mating windows 345, 350. The assembly of interlocking members 110, 200 may, at this point, be advanced, retracted or adjusted along the length of the suture extensions 50, 60. When final securement is desired, the interlocking members 110, 200 are compressed and the suture 50, 60 is trapped in a convoluted and tortuous pathway and partially compressed by somewhat resilient material or by generous radii and tolerances. There are locking or latching features 316, 321 located upon the standing portions 315, 320 that are sized and configured to mate with receiving portions 318 adjacent to or within the window portions 345, 350 of the base 250. The locking or latching portions 316, 321 resist the forces applied by the suture 50, 60 to pull the interlocking members 110, 200 apart while the protrusions 160, 170, 260, 270 resist the forces applied by the suture 50, 60 in an opposite direction. The forces that suture 50, 60 apply to the assembly are threefold and may be addressed separately.

First, the force that suture 50, 60 apply in opposing directions at a distal end may be resisted by protrusions 160, 170, 260, 270 and is maintained as the protrusions extend in a locked or latched relationship through mating holes 180, 190, 280, 290, respectively, in the adjacent interlocking member. Second, the force applied by the suture in traction which attempts to force either of the interlocking members apart may be resisted by the locking or latching features 316 of the standing portions as they engage an undercut edge 318 within the windows 345, 350 of the mating members. Third, the force that suture 50, 60 apply to the assembly may be determined by the traction of suture 50, 60 have upon the material surface of the interlocking members. In addition, the tortuous and convoluted pathway 376 requires the suture to pass over a plurality of radii 375 and material interfaces. The materials from which the interlocking members are made are chosen to cooperate to provide appropriate traction and durability. Material choices may include plastics that are of the same genus as the suture itself. This will take advantage of the tractive relationship between like plastic materials. Other choices may include biodegradable plastics such as polyglycolic acid or polylactate materials that have a rate of absorption commensurate with the suture used. Metal may be used in some applications and may include stainless steel, titanium or rare metals. In alternate embodiments, a combination of metal and plastic may be used where plastic is chosen for its value as an atraumatic interface and metal is chosen for its radiopacity or durability.

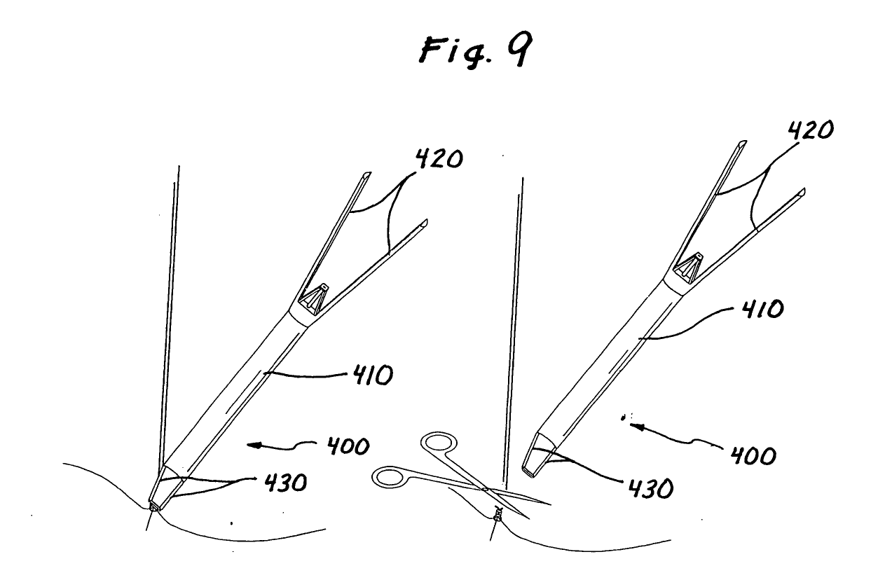

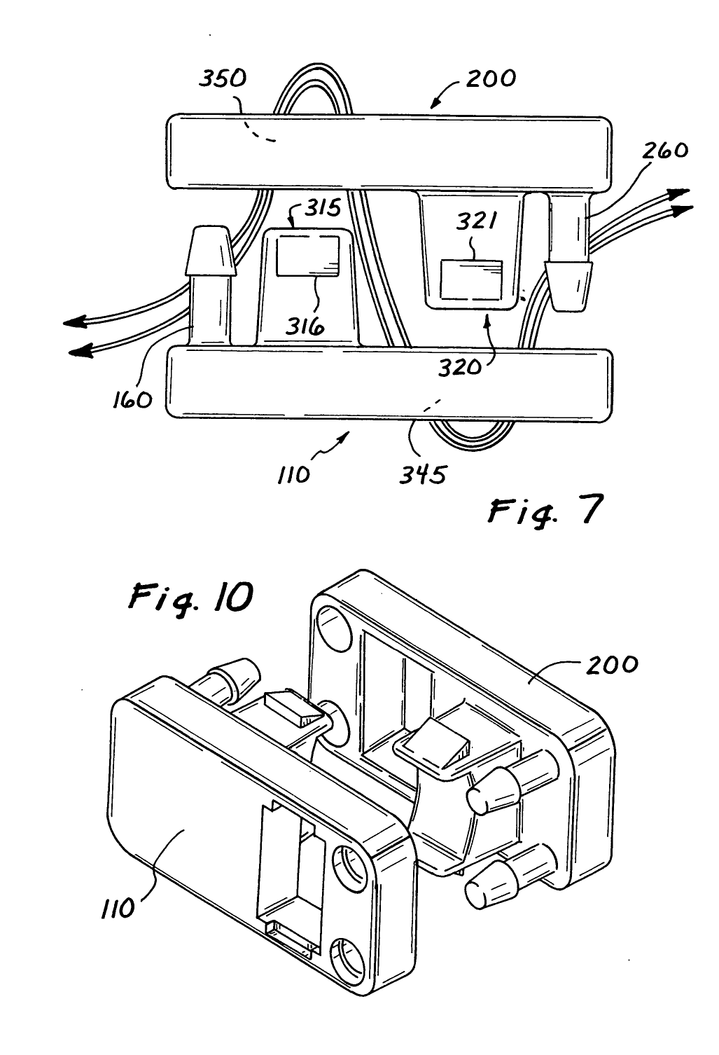

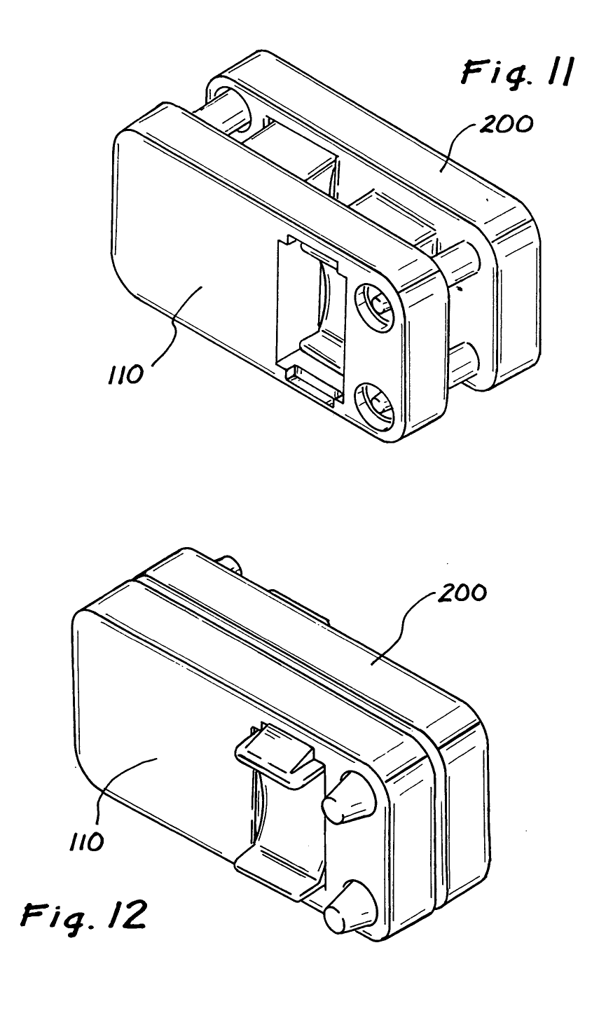

Referring to FIG. 9, there is shown a perspective view of a suture clinch and clinch applier 400 operatively positioned for use in a patient. The clinch applier 400 comprises an elongate body 410, a handle 420, and a channel 430 for advancing pairs of interlocking members 110, 200 upon suture extensions 50, 60. The individual interlocking members 110, 200 are loaded into the channel 430 of the elongate body 410 in an opposing, mating arrangement and are advanced as needed into the movable jaw(s) (not shown). The individual interlocking members 110, 200 are held in a separated relationship (see, e.g., FIG. 10) that allows suture extensions 50, 60 to be easily placed within the interlocking members 110, 200. When the suture is within the confines of the interlocking members 110, 200, the jaw(s) are closed to a second condition (see, e.g., FIG. 11) which forces the suture 50, 60 into a low profile tortuous path that allows the interlocking members 110, 200 to be positioned along the length of the suture extensions. When proper placement is achieved, the jaws are closed to a third and final condition (see, e.g., FIG. 12) that latches and locks the interlocking members 110, 200 together and fully traps the suture extensions 50, 60 within the interlocking members 110, 200.

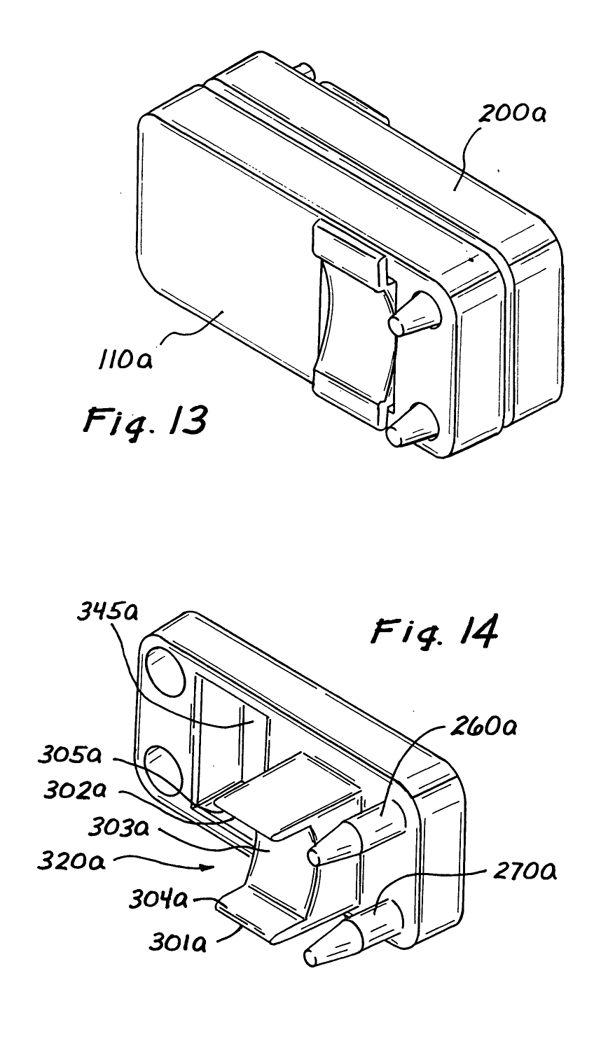

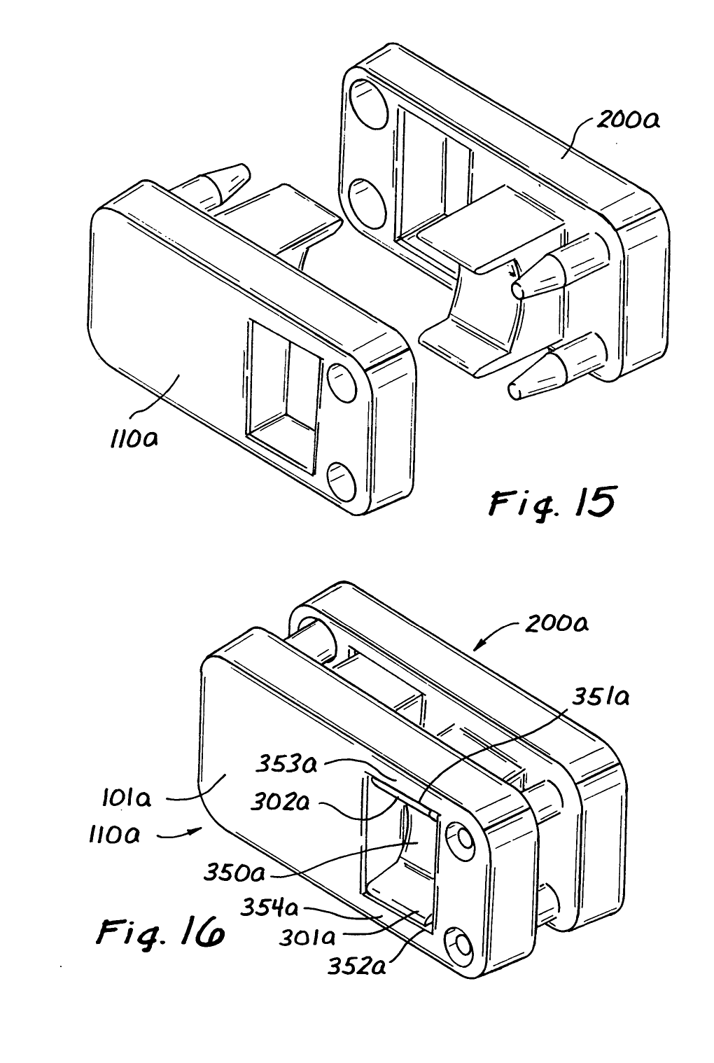

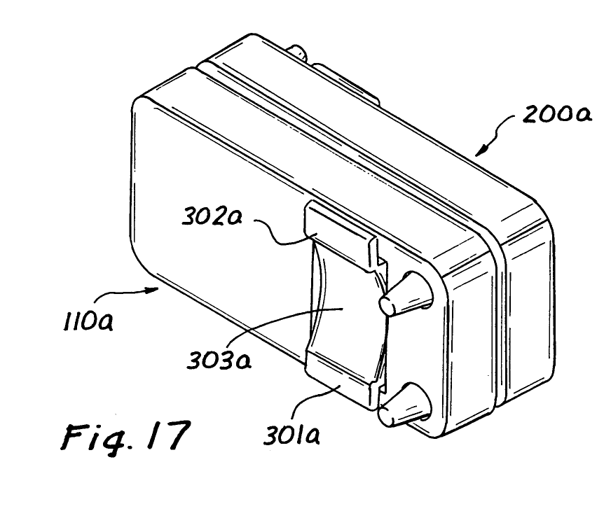

FIGS. 13-17 illustrate an alternate embodiment of the interlocking members of the invention comprising of a metallic material. In this embodiment, structural elements which are similar to those previously discussed will be designated by the same reference numeral followed by the lower case letter “a”. The metallic material may be a soft metal such as titanium, silver, gold, stainless steel, aluminum or the like. An advantage of using metallic interlocking members is they may eliminate the use of undercuts and barbs/latches altogether as compared to plastic interlocking members. The metallic interlocking members may include an extension of protrusions 260a, 270a and an extension of raised side portions 301a, 302a of standing member 320a. As the raised side portions 301a, 302a pass through window 350a of mating member 110a, they are deformed by a feature within the jaw channel. The deformation or deflection of the raised side portions 301a, 302a is preferably toward the sides of the interlocking members 110a, 200a and away from suture path 303a. As the interlocking members 110a, 200a are fully compressed upon included suture extensions 50a, 60a, the raised side portions 301a, 302a are folded over side edges 351a, 352a and flattened against the back 101a of the mating member 110a in opposing regions 353a, 354a adjacent to the window 350a of the mating member 110a.

This arrangement of folded extensions locks the interlocking members 110a, 200a together and maintains appropriate pressure upon included suture extensions. This alternate embodiment is most appropriate for soft, malleable metals such as titanium. The use of metals, especially rare metals, would yield a suture securement that is radiopaque and therefore easily seen by standard viewing equipment.

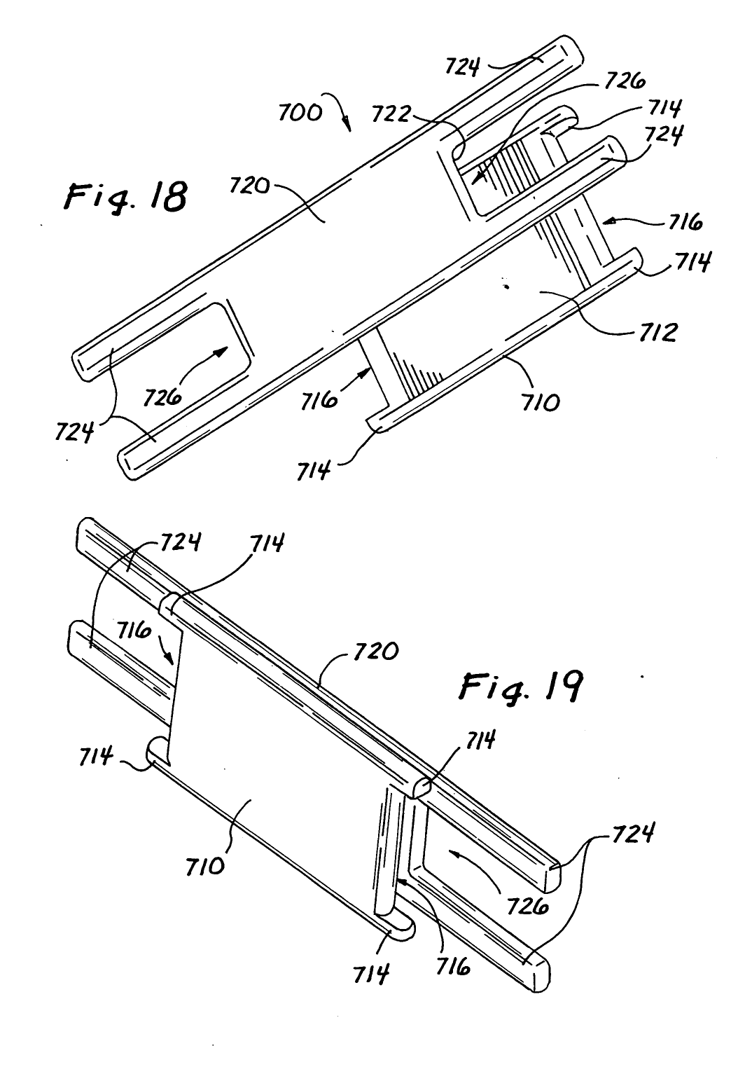

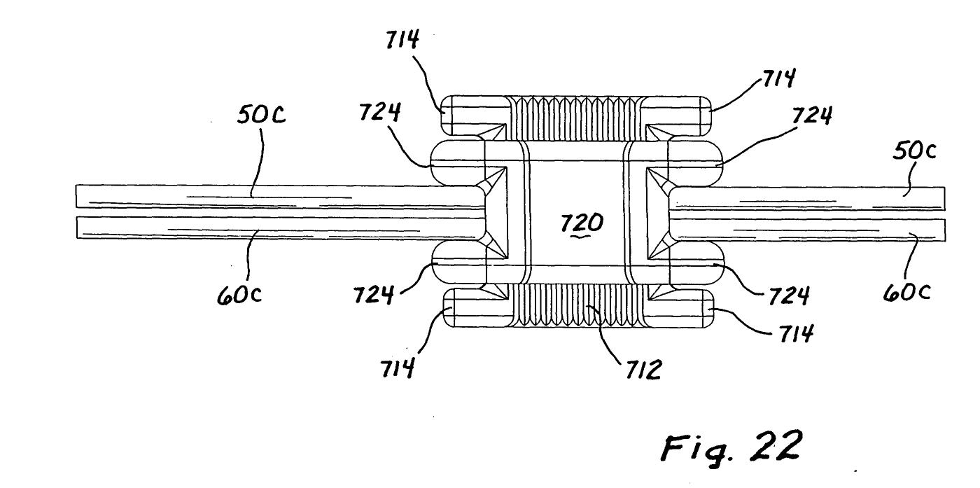

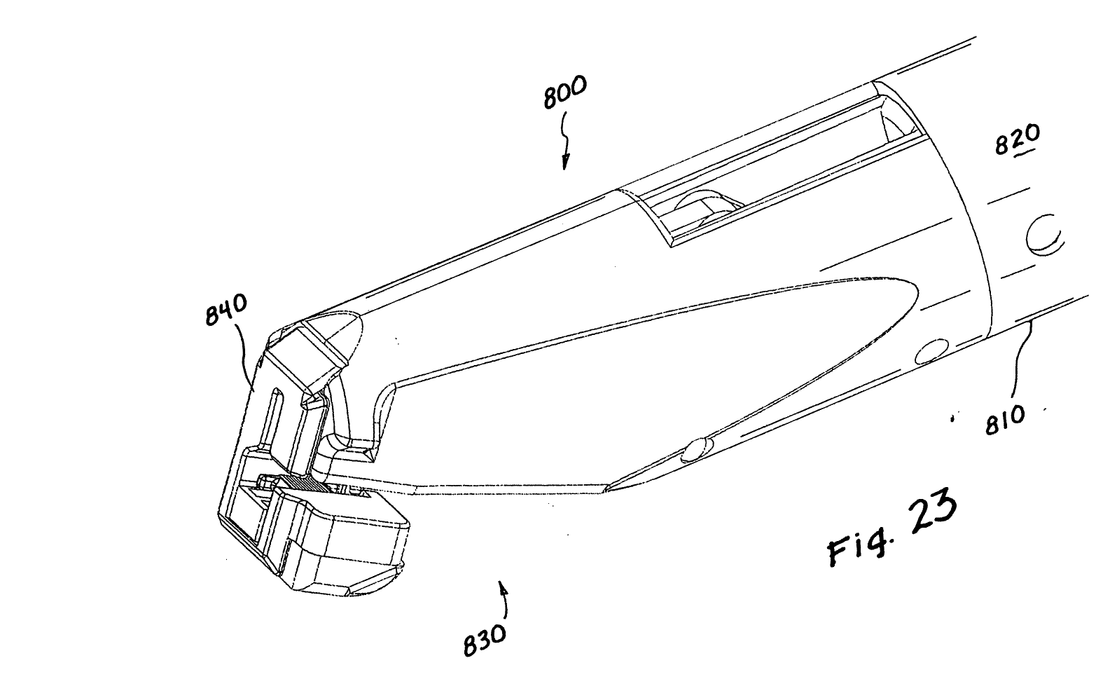

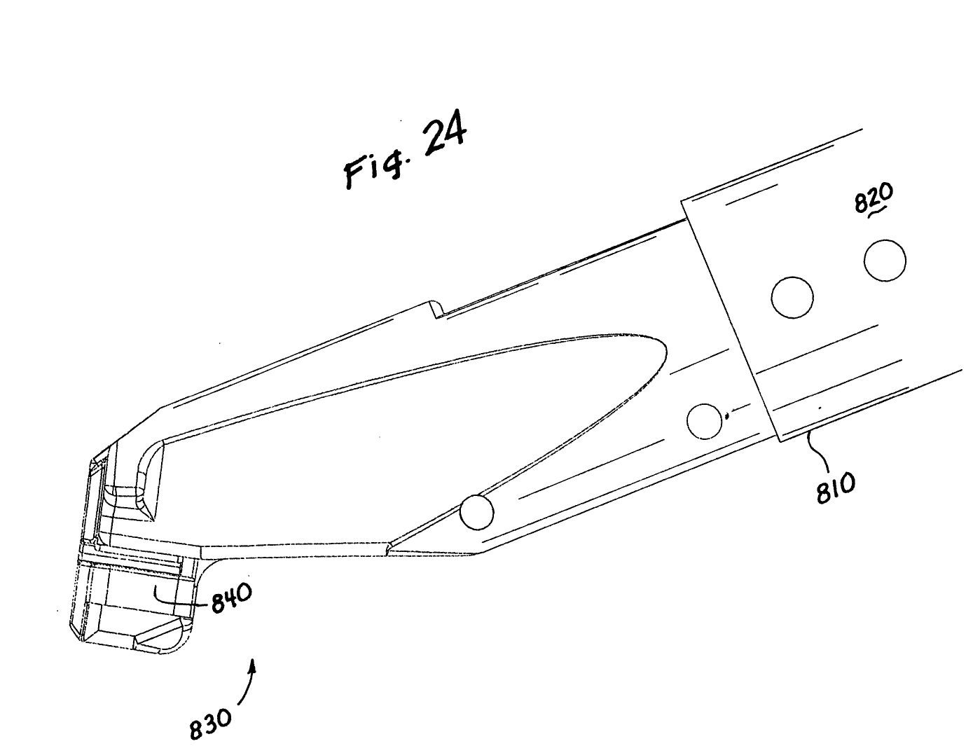

FIGS. 18-22 illustrate a suture clinch 700 in accordance with another embodiment of the invention in a flat form. The suture clinch 700 comprises an assembly of two individual components, a first component 710 and a second component 720. The first and second components are sized and configured to fit together in a mating relationship that entraps at least a portion of material between tractive faces 712, 722 of the first and second components 710, 720, respectively. Subsequent folding of the second component 720 over the first component 710 provides secure entrapment of material between the tractive faces 712, 722 of the first and second components.

The first component 710 may comprise, in a preferred embodiment, a first substantially flat form having a length, a width and a thickness. Each end, associated with the length, may have a recessed central portion 716 that leaves two extensions 714 along the sides of each end. At least one of the faces associated with the width and length has an interrupted surface or other traction-enhancing feature. A preferred embodiment comprises a traction-enhancing portion that is raised above the general surface of the first component. The raised component may comprise a surface that resembles the face of a “double-cut” flat file. The raised traction-enhancing surface is sized and configured to engage surgical suture and hold it securely in place without damaging or challenging the suture.

The second component 720 may comprise, in a preferred embodiment, a second substantially flat form having a length, a width and a thickness. Each end, associated with the length, comprises a pair of elongate extensions 724 and a recessed central portion 726. The elongate extensions 724 are sized and configured to fit between the end extensions 714 of the first component 710 and within the recesses 716 associated with the length of the first component 710 when the extensions 724 of the second component 720 are folded over the first component 710. In the mated and folded condition, the recesses 726 associated with the length of the second component 720 provide an exit path for suture entrapped between the faces of the first and second components 710, 720. The opposing face 722 of the second component 720 may have a traction-enhancing feature that compliments the traction-enhancing feature of the first component 710.

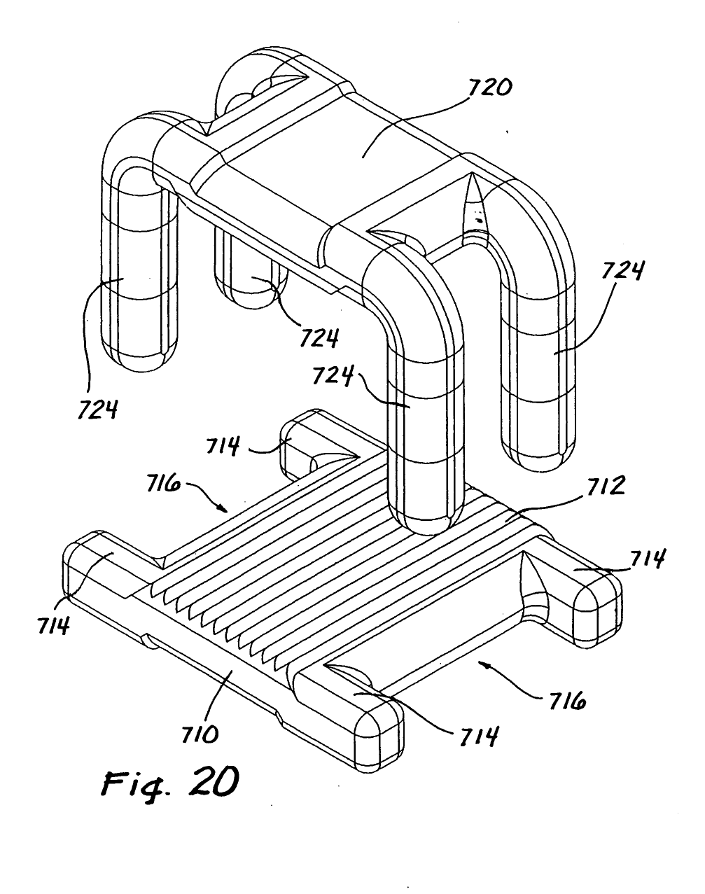

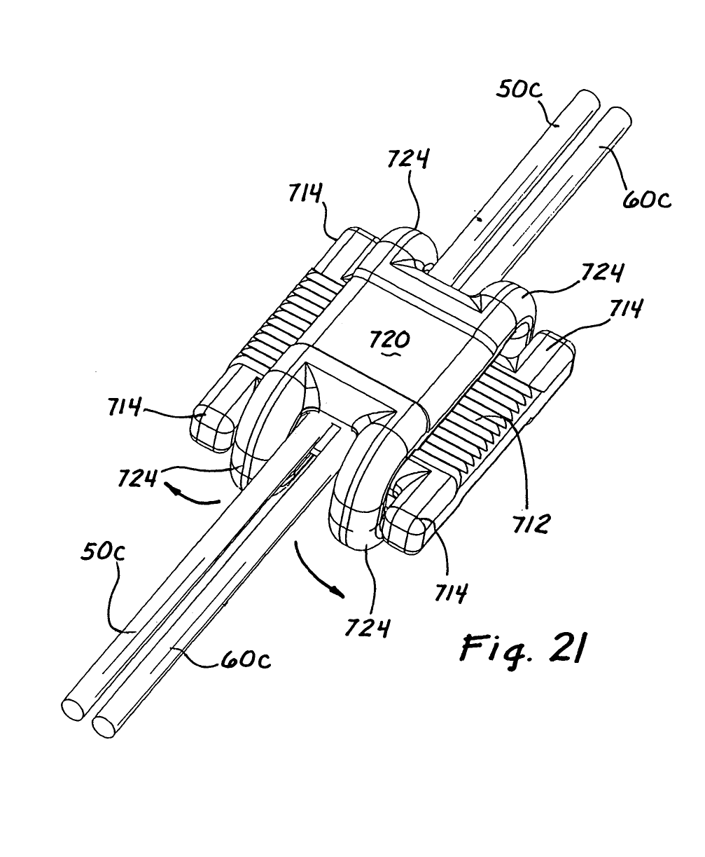

Referring to FIGS. 20 and 21, the second component 720 is seen in a first-folded condition. The first-folded condition is sized and configured to provide guidance for suture extension 50c, 60c to a preferred location within and between the two mating components 710, 720. In addition, the first-folded extensions 724 provide guidance and alignment between the two components 710, 720 prior to final assembly and final folding of the second extensions over the first component 710.

The two mating components 710, 720 of the invention may be used to entrap a portion of surgical suture or other elongate material by placing the suture or material between the two components 710, 720, and subsequently approximating the two components and further folding the extensions of the second component 720 over the first component 710. The recessed region 726 associated with the second component 720 is configured with sufficient material radii that the exposed edges of the material do not “notch,” or otherwise abrade or damage the delicate suture material.

It is of particular note that the interlocking nature of the first and second components 710, 720 of the invention provides entrapment of surgical suture or other elongate material in a unique combination. First, the entrapped material is held between two opposing faces 712, 722 that may have traction-enhancing features. However, the entrapped material is not crushed to the extreme that would be required without the traction-enhancing features. It is important to provide stability and security to the entrapped material without providing excessive pressure or stress on the delicate material. Therefore, a raised crosscut pattern having very small, sharp details is preferred. The centrally located raised pattern allows the entrapped material to move within the recesses associated with the second component without abrasion. Any motion associated with suture extensions is isolated within the recessed region. It is also noteworthy that in most circumstances the forces applied to a suture securement are across the axis of the suture extensions. In other words, the forces are nearly always a “cross-pull”. Therefore, it is important that the suture extending from the application and into the securement contacts generous material radii so that it is not mechanically challenged. The mated components 710, 720 of the invention lightly compress the entrapped suture between traction-enhanced faces 712, 722 and provide rounded material edges that are sized and configured to maintain the intrinsic strength of the suture as it exerts a “cross-pull” against the reinforced folded extensions 724 of the second component 720.

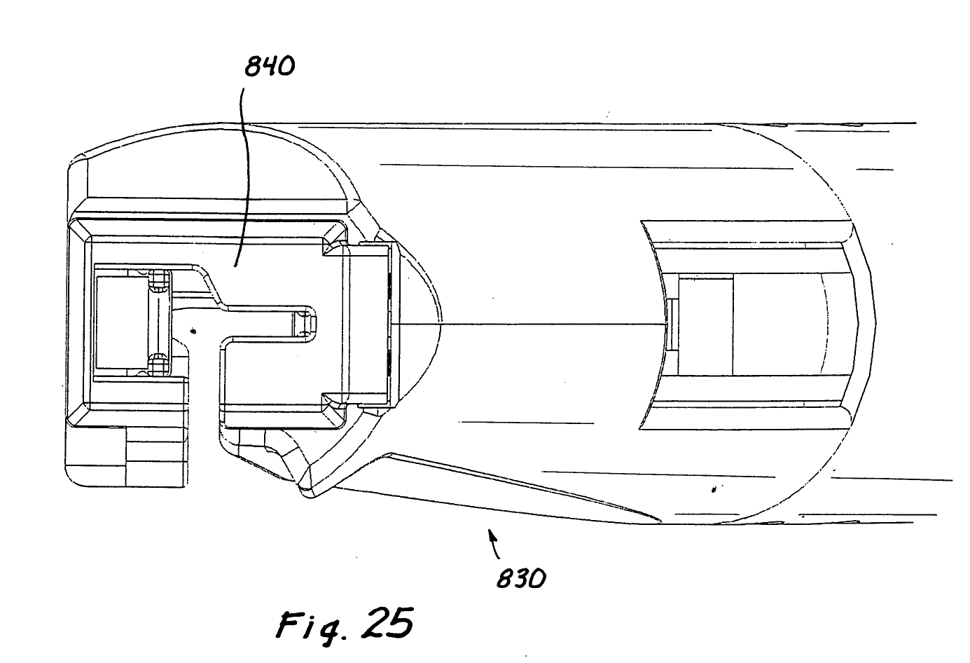

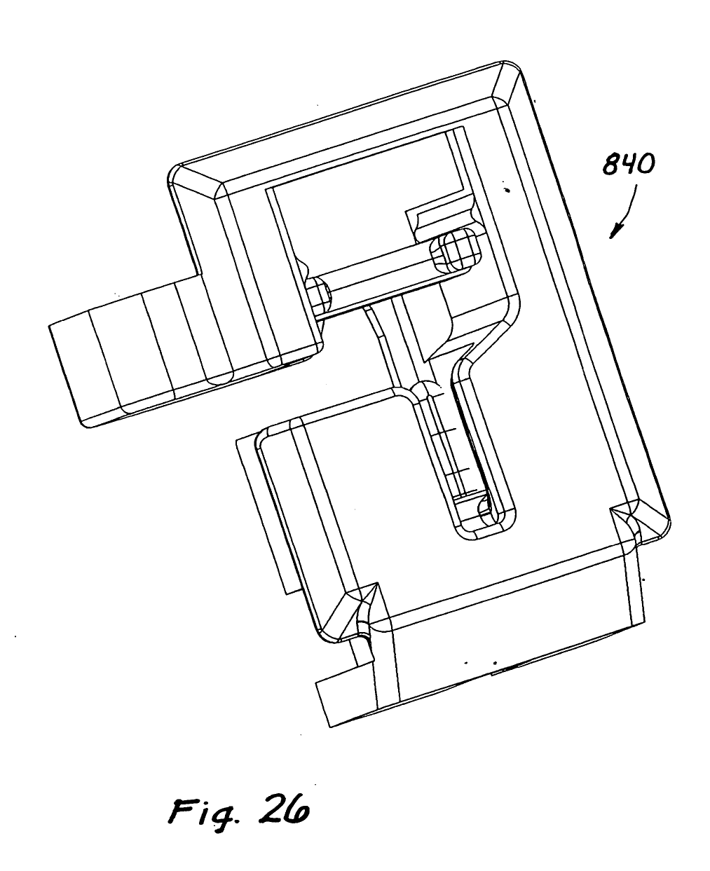

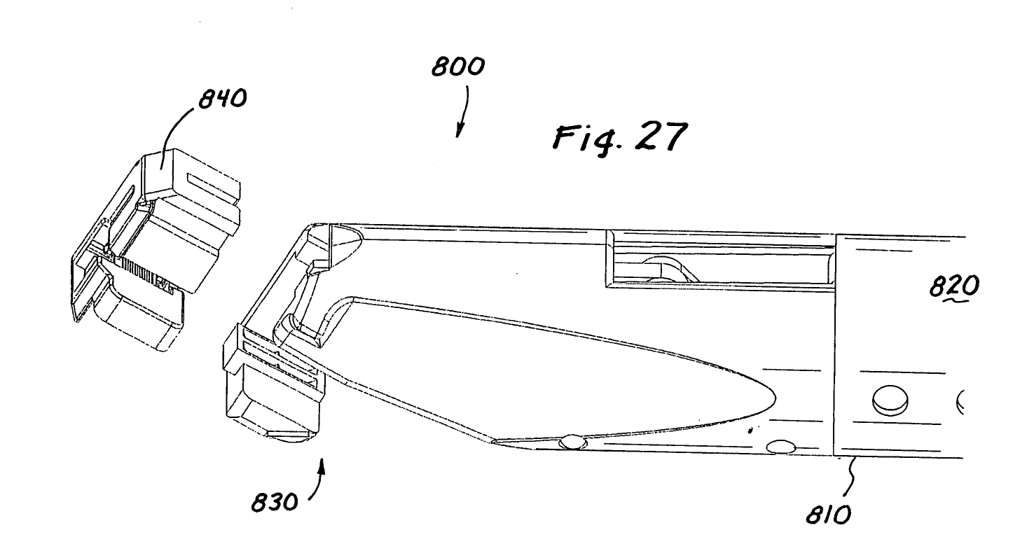

Referring to FIGS. 23-27, an applier 800 for the suture clinch of the present invention is shown having an elongate body 810, a proximal end 820 and a distal end 830. A handle (not shown) is associated with the proximal end 820 that is sized and configured to be operated by a user. At least a jaw portion 840 is associated with the distal end 830 that is sized and configured to guide suture or other elongate material into a preferred position within the distal portion and subsequently between the first and second components 710, 720 of the invention. The jaw portion 840 comprises elements that compress the first and second components 710, 720 upon properly positioning material to an appropriate pressure and subsequently folding the first-folded length extensions 724 of the second component 720 over the first component 710. The action of the applier 800 locks the first and second components 710, 720 together over a portion of lightly compressed material.

The elongate body 810 or shaft portion may contain a supply of first and second components 710, 720 in a “ready to use” configuration. An alternate embodiment may include a “single-fire” construction where the first and second components 710, 720 are loaded into the distal end 830 individually or from a supply cartridge. Yet another embodiment contemplates the use of a pre-loaded cartridge having one or more pairs of first and second components 710, 720 that are arranged for deposit into the distal end 830 of the applier 800. Still another embodiment contemplates a self-contained jaw cartridge that may be connected to the distal end of an applier. Such a self-contained cartridge may comprise an arrangement of elements sized and configured to incrementally mate and compress the first and second components 710, 720. A self-contained cartridge may comprise a disposable, single-use device. In addition, a suture-cutting element may be associated with the compressive elements. A user may select to use the suture cutter to sever the excess suture after placement of the suture securement associated with the present invention.

An appropriate approach angle may be associated with the applier of the present invention that allows the suture securement device to be placed upon the trailing lengths of suture from the side. Therefore, it is not necessary to discard control of the suture ends in order to place the securement upon the suture. Once placed upon the suture extensions, the securement may be advanced along the suture extensions to the desired location and tension, compressed and subsequently released from the distal portion of the applier.

The following is an example of an application of the suture clinch 700 of the invention where soft titanium metal was chosen because of its sturdiness, strength and biocompatibility. The clinch 700 comprises a first component 710 and a second component 720 that are very small in overall size. The size may be determined by comparison to a standard surgical knot. As an example of size, the combined first and second components 710, 720 may be 0.150″ in length, 0.080″ in width and 0.080″ in folded thickness. This folded and applied size resembles a “four or five-throw” surgical square knot, a surgical “Tayside” knot or a surgical “Rueters” knot.

Laparoscopic surgery often requires a “slip-knot;” an advantage of the invention is it may be applied in the same manner as the “slip-knot”. The device may be placed upon the suture extension intracorporeally or extracorporeally and advanced to the appropriate position to secure a portion of suture as one would advance a slip-knot. The entrapment of suture extension between the first and second components 710, 720 of the invention is developed incrementally. The first increment comprises capture of the suture extensions 50c, 60c between opposing faces 712, 722 and between “first-folded” extensions 724 of the second component 720. The second increment comprises “light compressive engagement” of the suture extensions 50c, 60c to test placement of the device. The third increment comprises “complete folding of the first-folded extensions” of the second component 720. The fourth increment “releases” the mated and secured first and second components 710, 720.

It will be understood that many other modifications can be made to the various disclosed embodiments without departing from the spirit and scope of the invention. For these reasons, the above description should not be construed as limiting the invention, but should be interpreted as merely exemplary of preferred embodiments.

1. A securing mechanism for securing a pair of free ends of a suture, comprising:
a first interlocking member; and
a second interlocking member operable of mating with said first interlocking member,
wherein the first and second interlocking members provide
a first position for capturing the suture ends,
a second position for guiding and aligning the suture ends to a preferred location along a tortuous path within and between the first and second interlocking members, and
a third position for frictionally engaging the suture ends to provide secure entrapment of the suture, and
wherein each of said interlocking members has
at least one protrusion and at least one mating hole, and
a standing portion and a mating window.

2. The securing mechanism of claim 1, wherein the protrusions are cylindrical and are sized and configured to match opposing mating holes.

3. The securing mechanism of claim 1, wherein the protrusions further comprise barbs or have increased end diameters to engage opposing mating holes in a fixed relationship when fully mated.

4. The securing mechanism of claim 1, wherein the mating windows are sized and configured to receive opposing standing portions.

5. The securing mechanism of claim 1, wherein the standing portions and mating windows are sized and configured to engage and confine the suture ends.

6. The securing mechanism of claim 1, wherein the assembly of the interlocking members may be advanced, retracted or adjusted along the length of the suture.

7. The securing mechanism of claim 1, wherein the standing portions further comprise locking or latching features.

8. The securing mechanism of claim 7, wherein the mating windows further comprise receiving portions to mate with the locking features of the standing portions.

9. The securing mechanism of claim 1, wherein the interlocking members are formed of plastics of the same genus as the suture.

10. The securing mechanism of claim 1, wherein the interlocking members are formed of metal.

11. The securing mechanism of claim 10, wherein the metal includes stainless steel, titanium, silver, gold and aluminum.

12. The securing mechanism of claim 10, wherein the metal is malleable.

13. The securing mechanism of claim 1, wherein the interlocking members are formed of plastic and metal.

14. A securing mechanism for securing a pair of free ends of a suture, comprising:
a first substantially flat form component having a length and a width, each end associated with the length comprising a pair of end extensions and a recessed portion, and at least one of the faces of the first component has a traction-enhancing feature to engage the suture;
a second substantially flat form component operable of mating with said first component having a length and a width, each end associated with the length comprising a pair of elongate extensions and a recessed central region, wherein the elongate extensions are sized and configured to fit between the end extensions and recessed portions of the first component when the elongate extensions of the second component are folded over the first component,
wherein the first and second components provide
a first position for capturing the suture ends,
a second position for guiding and aligning the suture ends to a preferred location within and between the first and second components, and
a third position for frictionally engaging the suture ends to provide secure entrapment of the suture.

15. The securing mechanism of claim 14, wherein at least one of the faces of the second component has a traction-enhancing feature to engage the suture.

16. The securing mechanism of claim 15, wherein the faces of the first and second components having traction-enhancing features are opposed to one another.

17. The securing mechanism of claim 14, wherein in the folded condition, the recessed central regions of the second component provide an exit path for the suture entrapped between the faces of the first and second components.

18. The securing mechanism of claim 14, wherein the recessed central regions of the second component provide rounded material edges so as not to abrade or damage the suture material.

19. The securing mechanism of claim 14, wherein the traction-enhancing feature is a raised crosscut pattern above the general surface of the first component.

20. The securing mechanism of claim 14, wherein the traction-enhancing feature comprises a surface resembling the face of a “double-cut” flat file.

21. The securing mechanism of claim 14, wherein the first and second members are formed of at least one of metal and plastic.
